(19) United States
(12) Patent Application Publication
(10) Pub. No.: US 2009/0012474 A1
(43) Pub. Date: Jan. 8, 2009

(54) Syringe Driver Monitoring Means

(76) Inventor: Robert Senior, Denbighshire (GB)

Correspondence Address: CHARLES N. QUINN;FOX ROTHSCHILD LLP, 2000 MARKET STREET, 10TH FLOOR, PHILADELPHIA, PA, 19103, US

(21) Appl. No.: 11/664,972

(22) PCT Filed: Oct. 12, 2005
(86) PCT No.: PCT/GB05/03932

§ 371 Date: Sep. 5, 2008

(30) Foreign Application Priority Data
Oct. 15, 2004 (GB) 0422884.7

(51) Int. Cl. A61M 5/20

(52) U.S. Cl. 604/154

(57) ABSTRACT

A mechanism for receiving a thumbplate of a syringe within a syringe driver, the mechanism comprising a first fixed jaw (6) and a second moveable jaw (8) for receiving the thumbplate therebetween, the moveable jaw being coupled to a marker (16) in contact with a position-defining component (18).

The present invention relates to a syringe driver monitoring means.

Syringe drivers or pumps are well known in the art. They are small, lightweight, battery operated machines that are designed to administer from a syringe subcutaneous infusions of a prescribed amount of medication over a given period. A syringe driver assembly basically consists of the machine itself for driving a plunger of a syringe, a syringe containing the medicine to be administered and a thin piece of tubing connected to the syringe which has a needle at the end of it. Generally, both the machine and the syringe are contained within a housing to increase the portability of the device.

The drive mechanism for driving the plunger through the syringe barrel to dispense medication generally consists of a motor, gears and a threaded shaft. The motor causes rotation of the threaded shaft which, via an actuator attached thereto, effects movement of the plunger. Once the required medication has been dispensed, it is necessary to manually reset the syringe driver by pulling back the actuator and syringe plunger to the required degree. Conventionally, this is achieved by the provision of two half nuts around the threaded shaft, the manual disengagement of which enables the actuator to be moved back to the end of the shaft to allow the plunger to be reset. This procedure normally requires the use of two hands.

It is important for a syringe driver assembly to be able to recognize the type and size of syringe that is placed in the housing so that the correct dosage and type of medication may be administered. It is also desirable for the movement of the plunger to be monitored as it dispenses fluid from the syringe so that the amount of medication being administered to the patient may be recorded. Conventionally, a linear potentiometer or optical encoder is used to monitor movement of the plunger along the longitudinal axis of the unit. However, these readings are not always that accurate. The cradle of the syringe driver assembly may be dimensioned only to receive a standard size of syringe to ensure that the correct dosage and type of medication is administered. However, it is preferable to be able to use any size and type of syringe within the same syringe driver housing.

It is an aim of the present invention to provide a syringe driver assembly that can recognize syringes of different types and sizes.

Another aim of the present invention is to provide enhanced monitoring of a syringe as it dispenses fluid by means a syringe driver assembly.

Yet a further aim of the present invention is to provide a syringe driver assembly that is easier to load.

Accordingly, a first aspect of the present invention provides a mechanism for receiving an end of moveable member, the mechanism comprising a first fixed jaw and a second moveable jaw for receiving the end of the member therebetween, the moveable jaw being coupled to a marker in contact with a position-defining component.

The mechanism is generally employed in a syringe driver assembly, wherein the moveable member is a syringe plunger, the end of the plunger comprising a thumbplate. To this end, a second aspect of the present invention provides a syringe driver assembly comprising driver means for imparting controlled translational movement to a syringe plunger to drive the plunger through a syringe barrel, the driver means including a motor that causes rotation of a shaft which is linked to an actuator for contacting the plunger, characterised in that actuator includes a mechanism for receiving a thumbplate of the plunger, the mechanism comprising a first fixed jaw and a second moveable jaw for receiving the thumbplate therebetween, the moveable jaw being coupled to a marker in contact with a position-defining component.

In this manner, closure of the moveable jaw over the thumbplate causes a corresponding movement in the same plane of the marker that, via the position-defining component, may be used to determine the diameter of the thumbplate. Generally, this movement is along the y axis of the syringe and driver assembly or in the vertical plane. Preferably, the marker is also able to monitor movement of the thumbplate in the x axis (generally along the horizontal plane) due to it being coupled to the jaws of the actuator that is moved by means of the shaft.

Preferably, the fixed jaw comprises a lower jaw and the moveable jaw comprises an upper jaw. It is preferable for the fixed jaw to be provided with a bevelled outer edge to assist in locating the member within a recess of the jaw. More preferably, the bevelled edge is formed by at least one generally V-shaped projection extending outwardly from the actuator wherein the apex of the V is remote from the actuator.

The upper jaw preferably comprises at least one bevelled inner edge to assist in retaining the member within a recess of the jaw. More preferably, this is provided by at least one generally V-shaped projection extending downwardly from the top of the actuator, wherein the apex of the V points towards the lower fixed jaw.

Preferably, the shaft is surrounded by a carriage that is linked to the actuator that carries the jaws for receiving the thumbplate of a syringe plunger. The shaft rotates and imparts translational movement to the carriage which causes a corresponding movement in the actuator which forces the plunger to move thereby dispensing medication from the barrel of the syringe.

Preferably, the marker is in the form of a stylus for contacting the position-defining component. The stylus may be capacitive or inductive pick up. However, preferably, the stylus forms a mechanical contact with the position-defining component only. It is preferred for the position-defining component to be in the form of a position transducer wherein a mechanical, non-electrical signal is converted into an electrical signal for mapping of the position of the stylus. More preferably, the position-defining component is in the form of a printed circuit board.

In a preferred embodiment of the present invention, opening of the jaws is linked to disengagement of the carriage from the shaft. Preferably, means for disengaging the carriage from the shaft to allow the carriage and actuator to move freely of the shaft causes the automatic lifting of the moveable jaw, for example by a cam and cam follower. It is preferable for the means for disengagement of the shaft to be activated by a switch that may be operated single-handedly. In this manner, operation of the switch releases the thumbplate of the syringe plunger from between the jaws of the actuator and allows the actuator to be re-set in position for loading of another syringe.

The movement of the actuator may be affected by means of a conventional motor-driven threaded shaft or by means of rotation of an unthreaded shaft carrying an odd number of bearings wherein alternate bearings are set at the same angle relative to the shaft and adjacent bearings are set at an opposing angle relative to the shaft. Preferably, a spring mechanism or cam follower is provided for manually disengaging one or more alternate bearings to enable the carriage to run freely of the shaft and/or to affect lifting of the moveable jaw.

Preferably, the syringe driver assembly is provided with further monitoring means in addition to the x y position marker. For example, an adjustable bracket or hook may be provided to clamp the barrel of the syringe into a cradle of the syringe driver. The position of the bracket may be used to determine the diameter of the syringe barrel. More preferably, the bracket or hook is spring loaded with means being provided for determination of the amount of extension in the spring that may be used to calculate the diameter of the syringe barrel.

One or more sensors may also be provided in the cradle that receives the barrel of the syringe for sensing placement of a syringe in the cradle. Preferably, at least two sensors, for example in the form of switches, are provided at predetermined positions that correspond to the location of an annular flange provided at the end of a syringe barrel. The location of the flange once inserted into the cradle of the syringe driver will depend upon the size and type of syringe inserted and may be pre-determined for standard types and sizes of syringe. In this manner, loading of a syringe into the cradle will result in the flange abutting one of the sensors. The sensor will be able to determine the size and/or type of syringe received. This may be cross-checked with one or more of the other monitoring means, such as that which measures the diameter of the thumbplate and/or the diameter of the syringe barrel.

Preferably, the monitoring means are used in conjunction with a look-up table of known syringe types. This may be used to ensure that the correct syringe type is inserted, that it is inserted correctly and/or that the correct medication in the correct dosage is being administered. It is to be appreciated that the system and interpretation of the sensor outputs may be carried out using an on-board microprocessor and indeed this is preferred. The look-up table may be pre-programmed or programmable externally or by insertion of a memory card.

For a better understanding of the present invention and to show more clearly how it may be carried into effect reference will now be made by way of example only to the accompanying drawings in which:

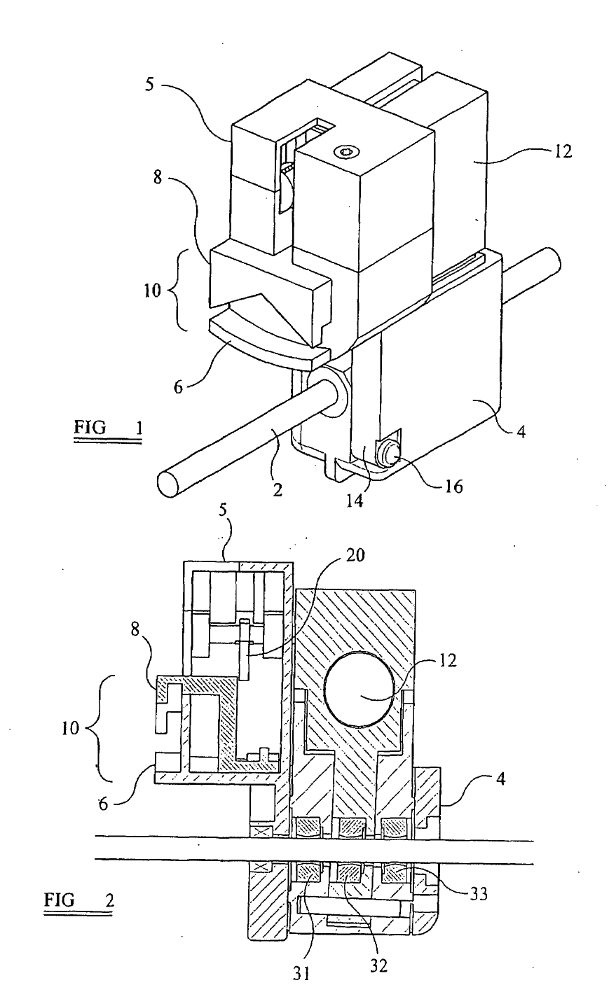

FIG. 1 is a perspective view of a mechanism for receiving a thumbplate of a syringe plunger according to one embodiment of the present invention;

FIG. 2 is a cross-sectional view of the mechanism shown in FIG. 1;

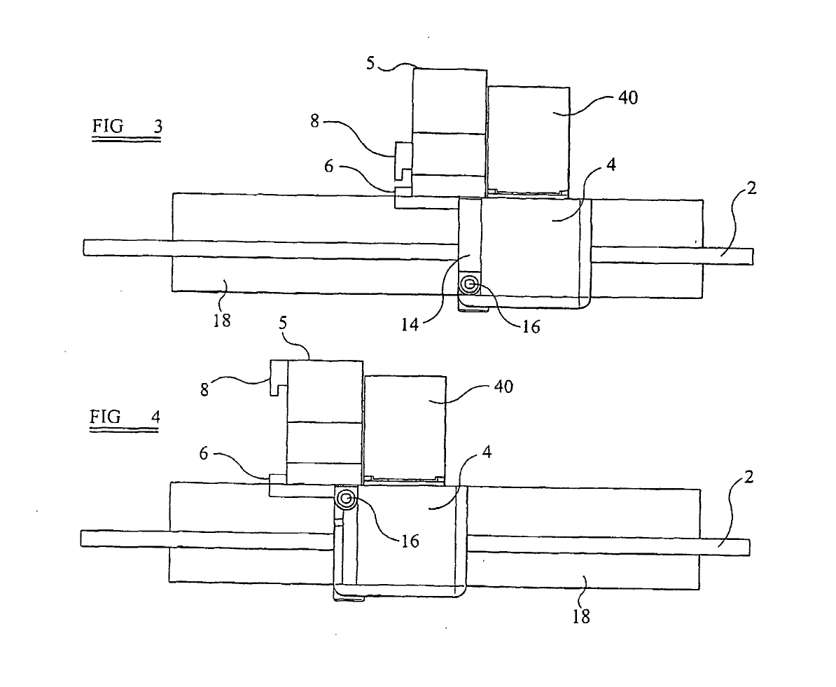

FIG. 3 is a side view of the mechanism shown in FIG. 1, shown with the mechanism closed;

FIG. 4 is a side view of the mechanism shown in FIG. 1, shown with the mechanism open;

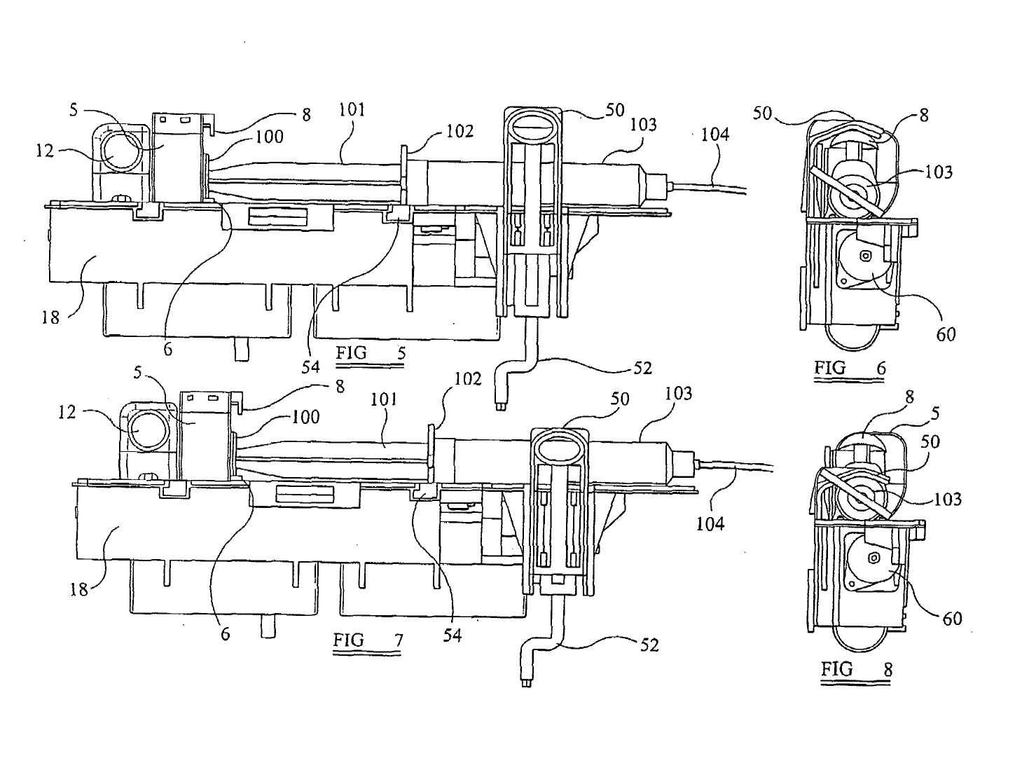

FIG. 5 is a side view of a syringe driver assembly containing the mechanism shown in FIG. 1 and additional monitoring means, including a bracket shown in its raised position;

FIG. 6 is an end view of the syringe driver assembly shown in FIG. 5;

FIG. 7 is a side view of the syringe driver assembly shown in FIG. 5 but with the bracket in its lowered position; and

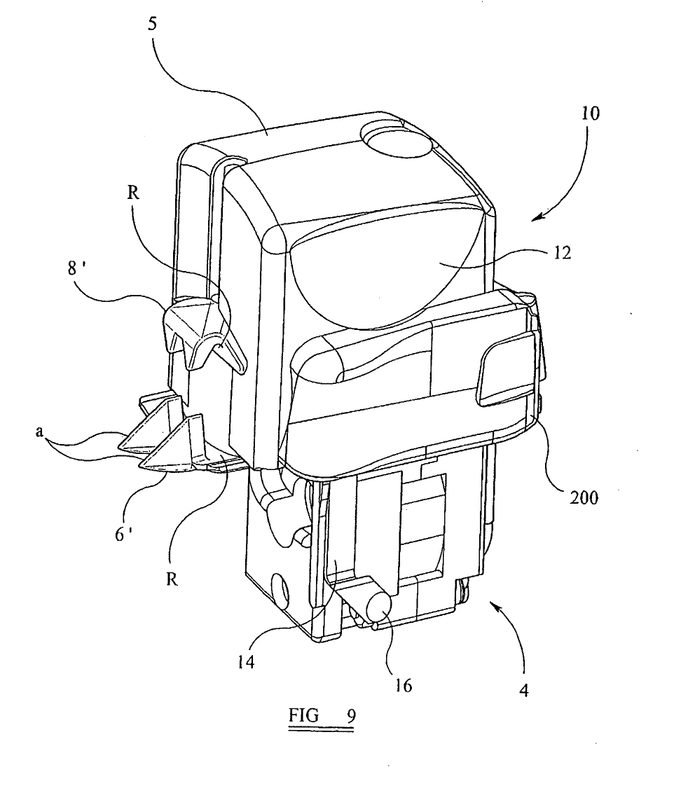

FIG. 8 is an end view of the syringe driver assembly shown in FIG. 7; and

FIG. 9 is a perspective view of a mechanism for receiving a thumbplate of a syringe plunger according to another embodiment of the present invention.

Referring to the accompanying drawings, a mechanism for receiving a thumbplate of a syringe plunger according to one embodiment of the present invention is illustrated (FIGS. 1 to 4), together with its incorporation into a syringe driver assembly (FIGS. 5 to 8). The mechanism 10 is carried on an actuator 5 that is connected to a carriage 4 mounted on a motor-driven shaft 2. The mechanism 10 comprises a fixed bottom jaw 6 and a moveable upper jaw 8. The upper jaw is linked by a member 14 that is able to move vertically within the actuator 5 to a marker 16, in the form of a stylus. The member, in its lowered position, lies within a recess provided in the front of the carriage 4. The stylus provides a mechanical contact with a position-defining component 18 (see FIGS. 3 and 4) in the form of a PCB transducer, such as that described in the Applicant's co-pending application.

In use, the moveable jaw 8 is lifted upwardly and a thumbplate 100 of a syringe plunger 101 (see FIGS. 5 to 8) is inserted between this and the lower fixed jaw 6. The actuator is pressed downwardly so that the upper jaw 8 abuts the top of the thumbplate thereby retaining the plate between the jaws. Pressing downwardly on the actuator also causes a corresponding movement of the stylus in the y axis and this is mapped on the position-defining component 18. The position of the stylus may be used to determine to the diameter of the thumbplate which can be used to monitor the size and/or type of syringe that has been placed between the jaws.

Additionally, the carriage 4 is able to move along the shaft 2 by rotation of the shaft imparting translational movement thereto. This movement causes a corresponding movement of the actuator 5 that pushes the thumbplate in the direction of movement of the carriage thereby causing the plunger to dispense medication from the syringe barrel 103 through the tube 104 and into the patient. The degree of travel of the plunger is also monitored by means of the stylus 16 and the position-defining component 18 since the stylus moves a corresponding distance along the x or longitudinal axis of the assembly as the actuator and thumbplate. The position of the stylus along the x-axis can be used to monitor the amount of medication dispensed to the patient.

Means is also provided for releasing the carriage 4 from the shaft 2 to enable the carriage to be manually slid along the shaft to allow re-loading and re-setting of the syringe driver assembly. Preferably, disengagement of the carriage from the shaft is coupled to the lifting of the jaw 8. In the illustrated embodiment, the shaft 2 is unthreaded and rotated by means of bearings 31, 32, 33 that are offset with respect to the shaft, as described in the Applicant's co-pending UK Application No. 0328558.2. A push spring mechanism 12 is provided for disengagement of the middle bearing 32 from the shaft thereby enabling the carriage to slide along the shaft. The push spring mechanism is also linked to a cam 20 that, via a cam follower, causes the moveable jaw to be lifted upwardly thereby enabling the thumbplate to be removed from between the jaws. In this manner, the simple operation of the spring mechanism 12 allows the carriage to be reset, the removal of a syringe and a new one inserted single-handedly.

The syringe driver assembly illustrated in FIGS. 5 to 8 is provided with additional monitoring means to check the type of syringe inserted into the cradle of the assembly and monitor the movement of the syringe plunger and thereby measure the amount of fluid administered to the patient from the syringe barrel. A spring-loaded bracket or hook 50 is provided in the region of the assembly that receives the barrel 103 of the syringe. The bracket or hook can be lifted up (as shown in FIGS. 5 and 6) to allow the syringe to be inserted into the cradle and then lowered so that the curved part of the bracket abuts the upper side of the barrel (see FIGS. 7 and 8). The amount of extension in the spring when the bracket abuts the barrel is communicated to sensing means 52 that is able to determine from this the diameter of the syringe barrel that has been inserted into the cradle of the syringe driver.

Additionally, two sensors in the form of switches 54 are provided in the region of the cradle that receives an annular flange 102 of the syringe barrel. The location of the flange in the cradle depends upon the size and type of syringe and whether it has been installed correctly. The switches are located in predetermined positions that correlate to the positioning of a particular size and/or type of syringe. In this manner, activation of a switch by means of the flange may be used to determine and check the type and/or size of syringe placed in the cradle.

The syringe driver assembly according to the present invention preferably has all the aforementioned types of monitoring means. However, it is to be appreciated that not all types need be present. The aforementioned configuration, i.e having an x y map for the thumbplate, means for measuring the diameter of the syringe barrel and means for determining the position of the annular flange of the barrel, should be used in conjunction with a look-up table of known syringe types to facilitate determination of a number of syringe parameters, as explained below.

The diameters of the syringe barrel and the thumbplate of the plunger are characteristic of particular syringe types and/or sizes. Therefore, the syringe type and/or size may be read off a look-up table if certain of those values are known.

For example, if sensor 50 (barrel diameter) reads 22 mm and sensor 16 (thumbplate diameter) reads 16 mm, according to the fictitious look-up table shown below, the syringe is A-type.

TABLE

| | | | | | Maxi- | |
|---|---|---|---|---|---|---|
| | Barrel | Barrel | Capac- | Plunger | mum | |
| Syringe | length | Diameter | ity | diameter | draw | |
| Type | (mm) | (mm) | (ml) | (mm) | (mm) | Drug |
| A | 82 | 22 | 25 | 16 | 70 | Painkiller |
| B | 70 | 15 | 12 | 12 | 55 | Antibiotic |
| . . . | . . . | . . . | . . . | . . . | . . . | . . . |

When the syringe type is known, further syringe properties can also be known such as the volume of fluid in the syringe barrel.

The system can also be used to make checks for safety reasons, for example, if the sensor 52 reads the barrel as 30 mm and the plunger diameter as 26 mm, the syringe is either neither of type-A or type-B or is incorrectly installed (eg. Canted). Another safety check may be to monitor for parameter changes, eg. a change in the output of the sensor 52 may indicate that the syringe has been dislodged.

Moreover custom syringes may be used for certain drugs requiring specialised dose regimes. For example, a type-B syringe may be used to only to deliver a certain type of antibiotic whereas a type-A syringe may administer only a painkiller. The look-up table could carry that information and, for example, restrict the delivery rate (determined by the output of the motor) to reduce the likelihood of overdose.

The system of sensing and interpreting the sensor outputs is carried out using an on-board microprocessor. The took-up table may be pre-programmed or programmable externally or by insertion of a memory card. Known logic functions can be used to determine the state, safety and operation of the syringe driver in use.

FIG. 9 of the accompanying drawings illustrates a preferred embodiment of the mechanism for receiving a thumbplate of a syringe plunger according to the present invention. Identical features to those shown in FIGS. 1 to 8 are given the same reference numerals and only the differences will be discussed in detail. Again the mechanism 10 is carried on an actuator 5 that is connected to a carriage 4 mountable on a motor-driven shaft (not shown). The fixed bottom jaw 6′ is provided with two parallel generally V-shaped projections extending outwardly that provide a ramp “a” to assist in inserting the syringe plunger into the recess R. The moveable upper jaw 8′ is also provided with two substantially parallel generally V-shaped projections extending downwardly that assist in retaining the syringe plunger within the mechanism. The upper jaw may be moved by moving the lever 200 outwardly from the actuator 5. The lever 200 may be locked in position against the actuator to prevent movement of the upper jaw 8′.

1-29. (canceled)

30. A syringe driver assembly comprising driver means for imparting controlled translational movement to a syringe plunger to drive the plunger through a syringe barrel, the driver means including a motor that causes rotation of a shaft that is linked to an actuator for contacting the plunger characterised in that the actuator includes a mechanism for receiving a thumbplate of the plunger, the mechanism comprising a first fixed jaw and a second moveable jaw for receiving the thumbplate therebetween, the moveable jaw being coupled to a marker in contact with a position-defining component or monitoring the position of the thumbplate in two perpendicular directions wherein movement of the marker along a y axis or vertical plane of the syringe driver assembly is by means of the marker being coupled to the moveable jaw and movement of the marker in an x axis or horizontal plane of the syringe driver assembly is by means of the marker being coupled to the jaws of the actuator moveable by means of the shaft.

31. A syringe driver assembly as claimed in claim 30 wherein the shaft is surrounded by a carriage that is linked to the actuator.

32. A syringe driver assembly as claimed in claim 30 or 31 wherein the marker is in the form of a stylus for contacting the position-defining component.

33. A syringe driver assembly as claimed in claim 32 wherein the stylus comprises a capacitive or inductive pickup.

34. A syringe driver assembly as claimed in claim 32 wherein the stylus forms a mechanical contact only with the position-defining component.

35. A syringe driver assembly as claimed in claim 34 wherein the position-defining component is in the form of a printed circuit board.

36. A syringe driver assembly as claimed in claim 35 wherein the fixed jaw comprises a lower jaw having a bevelled outer edge to assist in locating the member in a recess (R) of the jaw.

37. A syringe driver assembly as claimed in claim 36 wherein the moveable jaw comprises an upper jaw having a bevelled inner edge to assist in locating the member in a recess (R) of the jaw.

38. A syringe driver assembly as claimed in claim 37 wherein opening of the jaws of the mechanism is linked to disengagement of the carriage from the shaft.

39. A syringe driver assembly as claimed in claim 38 wherein means for disengaging the carriage from the shaft causes automatic lifting of the moveable jaw.

40. A syringe driver assembly as claimed in claim 39 wherein a cam and cam follower is used to cause automatic lifting of the moveable jaw.

41. A syringe driver assembly as claimed in claim 40 wherein the means for disengaging the shaft is activated by a switch that is operable single-handedly.

42. A syringe driver assembly as claimed in claim 41 wherein movement of the actuator is affected by a motor-driven threaded shaft.

43. A syringe driver assembly as claimed in claim 41 wherein movement of the actuator is affected by means of rotation of an unthreaded shaft carrying an odd number of bearings wherein alternative bearings are set at the same angle relative to the shaft and adjacent bearings are set at an opposing angle relative to the shaft.

44. A syringe driver assembly as claimed in claim 43 wherein a spring mechanism or cam follower is provided for manually disengaging one or more alternate bearings to enable the actuator to run freely and/or to affect lifting of the moveable jaw.

45. A syringe driver assembly as claimed in claim 44 wherein further monitoring means is provided in addition to the marker.

46. A syringe driver assembly as claimed in claim 45 wherein an adjustable bracket or hook is provided to clamp the barrel of the syringe into a cradle of the syringe driver, the position of the loaded bracket being used to determine the diameter of the barrel.

47. A syringe driver assembly as claimed in claim 46 wherein the bracket or hook is spring-loaded with means for determination of the amount of extension in the spring to enable calculation of the diameter of the barrel.

48. A syringe driver assembly as claimed in claim 47 wherein one or more sensors are provided in a cradle of the syringe driver that receives the syringe barrel for sensing placement of the syringe in the cradle.

49. A syringe driver assembly as claimed in claim 48 wherein the sensors are in the form of switches.

50. A syringe driver assembly as claimed in claim 49 wherein at least two sensors are provided at predetermined positions that correspond to the location of an annual flange provided at an end of the syringe barrel whereby the location of the flange once inserted into the cradle will depend upon the size and/or type of syringe.

51. A syringe driver assembly as claimed in claim 50 wherein means is provided for cross-checking of the readings of the multiple monitoring means.

52. A syringe driver assembly as claimed in claim 51 wherein the monitoring means are used in conjunction with a look-up table of known syringe types.

53. A syringe driver assembly as claimed in claim 52 where the look-up table is pre-programmed or programmable externally or by insertion of a memory card.

54. A syringe driver assembly as claimed in claim 43 wherein the various outputs are interpreted using an on-board microprocessor.
